(19) United States
(12) Patent Application Publication
(10) Pub. No.: US 2007/0197966 A1
(43) Pub. Date: Aug. 23, 2007

(54) Safety syringe

(75) Inventors: Chien-Yuan Lee, Tamsui Town (TW); Chih-Cheng Kao, Taipei (TW)

Correspondence Address: COOPER & DUNHAM, LLP, 1185 AVENUE OF THE AMERICAS, NEW YORK, NY, 10036, US

(73) Assignee: Linfair Engineering (HK) Co., Ltd.

(21) Appl. No.: 11/358,328

(22) Filed: Feb. 21, 2006

(51) Int. Cl. A61M 5/00

(52) U.S. Cl. 604/110

(57) ABSTRACT

A safety syringe has a syringe body, a needle hub with a needle, a casing and a plunger. The syringe body has a front end, a neck and a notch. The neck is formed on the front end and has a proximal end, a distal end and an outer surface. The notch is formed around the outer surface of the neck near the proximal end. The needle hub is mounted around the distal end of the neck. The casing is a hollow tube, is mounted around the syringe body and has a rear end, an inner surface and an annular groove. The groove is formed in the inner surface near the rear end. The plunger is mounted slidably in the syringe body. After breaking the neck at the notch, the casing detaches from the syringe body. Therefore, a used needle will not prick a user.

### BACKGROUND OF THE INVENTION

1. Field of the Invention

The present invention relates to a syringe and especially to a safety syringe for protecting a user from being pricked by a contaminated needle.

2. Description of the Related Art

With modem developments in medical technology, people understand routes of infection more clearly. One of the routes of infection is contamination of blood in a person's circulation system. If a contaminated needle pricks a person, the contaminated needle can transfer infectious material to the blood and infect the person. Therefore, the used needles must be discarded carefully. However, used needles easily prick people when needle covers are placed over used needles.

### SUMMARY OF THE INVENTION

The objective of the present invention is to provide a safety syringe that can keep a used needle from pricking a person when a protective cap is placed over the used needle.

To achieve the foregoing objective, a safety syringe having a syringe body, a needle hub with a needle, a casing, a plunger and a head. The syringe body has a front end, a rear end, an outside diameter, a neck and a notch. The neck is formed on the front end and has a proximal end, a distal end and an outer surface. The notch is formed around the neck in the outer surface near the proximal end. The needle hub is mounted around the distal end of the neck. The casing is a hollow tube, is mounted around the syringe body and has a front end, a rear end, an inside diameter, an inner surface and an annular groove. The inside diameter of the casing corresponds to the outside diameter of the syringe body. The annular groove is formed in the inner surface near the rear end. The plunger is mounted in the syringe body and has a front end, a rear end, a tip and a head. The tip is formed on the front end. The head is formed on the rear end. After breaking the neck at the notch, the casing detaches from the syringe body. Therefore, a used needle will not prick a user because no protective cap needs to be placed on the needle.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view of a first embodiment of a safety syringe in accordance with the present invention;

FIG. 2 is an enlarged exploded perspective view of the safety syringe in FIG. 1;

FIG. 3 is an enlarged side view in partial section of the safety syringe in FIG. 1;

FIG. 4 is an enlarged operational side view in partial section of the safety syringe in FIG. 1 after pulling a plunger of the safety syringe from a casing of the safety syringe;

FIG. 5 is an enlarged operational side view in partial section of the safety syringe in FIG. 1 after breaking a neck of the safety syringe at a notch around the neck;

FIG. 6 is an operational side view in partial section of a second embodiment of the safety syringe in accordance with the present invention after breaking a neck of the safety syringe at a notch around the neck;

FIG. 7 is a side view in partial section of a third embodiment of the safety syringe in accordance with the present invention after breaking a neck of the safety syringe at a notch around the neck;

FIG. 8 is a side view in partial section of a fourth embodiment of the safety syringe in accordance with the present invention;

FIG. 9 is a side view in partial section of a fifth embodiment of the safety syringe in accordance with the present invention after breaking a neck of the safety syringe at a notch around the neck;

FIG. 10 is a side view in partial section of a sixth embodiment of the safety syringe in accordance with the present invention without a needle hub;

FIG. 11 is an operational side view in partial section of the safety syringe in FIG. 10 after pulling a plunger of the safety syringe from a casing of the safety syringe;

FIG. 12 is a side view in partial section of the safety syringe in FIG. 10 after breaking a neck of the safety syringe at a notch around the neck;

FIG. 13 is an operational perspective view in partial section of a seventh embodiment of the safety syringe in accordance with the present invention after pulling a plunger of the safety syringe from a casing of the safety syringe; and

FIG. 14 is a side view in partial section of an eighth embodiment of the safety syringe in accordance with the present invention after breaking a neck of the safety syringe at a notch around the neck.

### DETAILED DESCRIPTION OF THE PREFERRED EMBODIMENTS

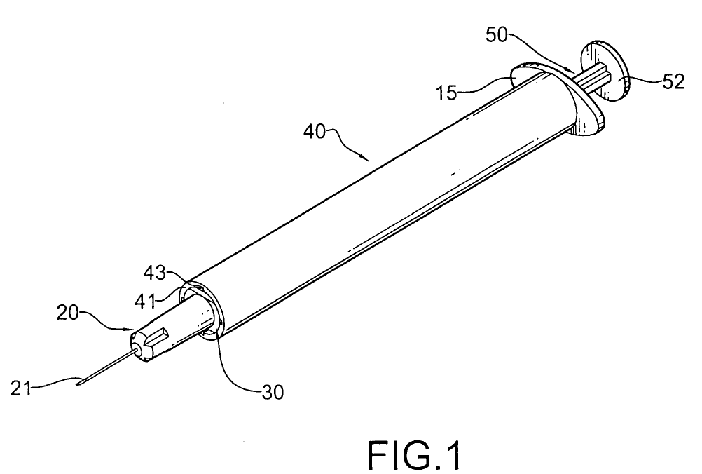

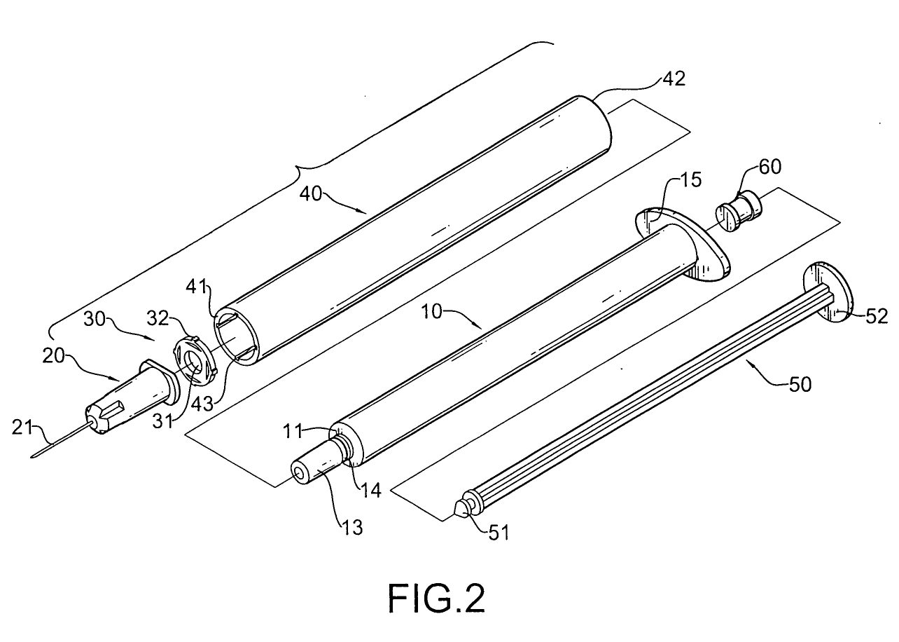

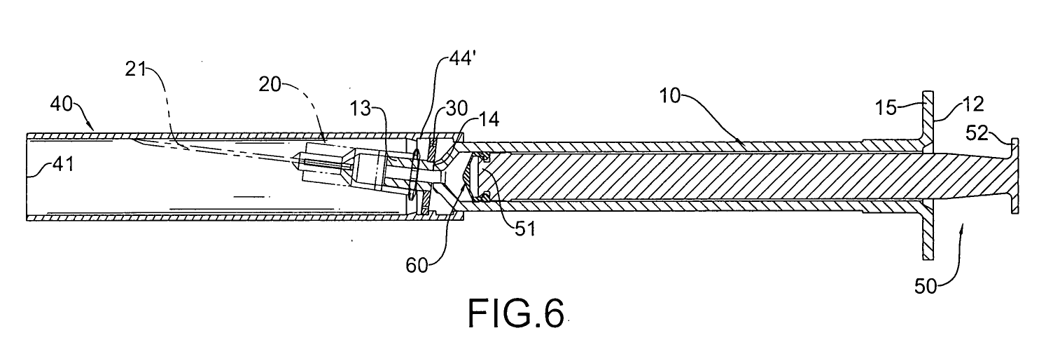

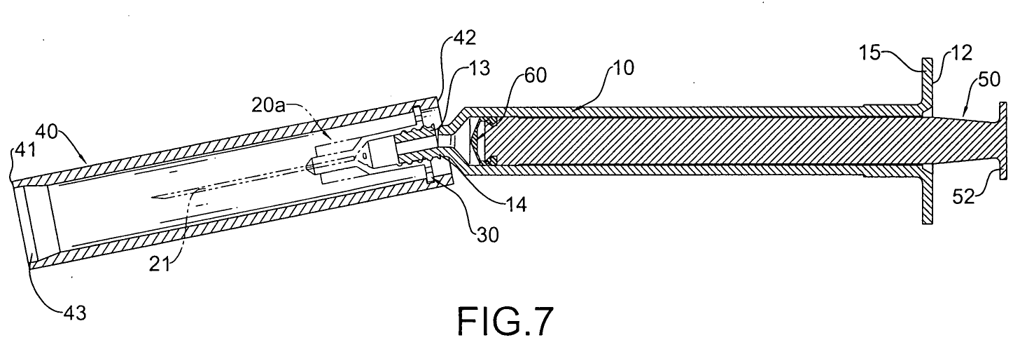

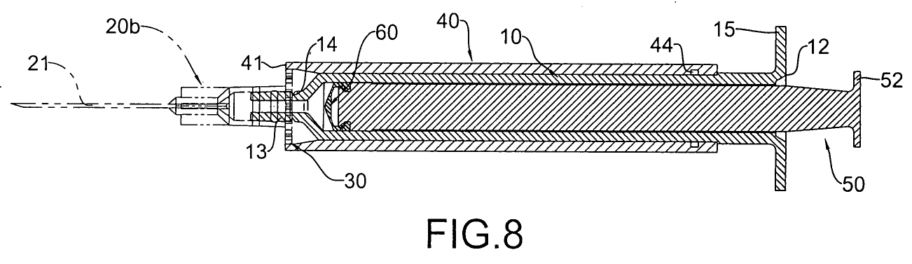

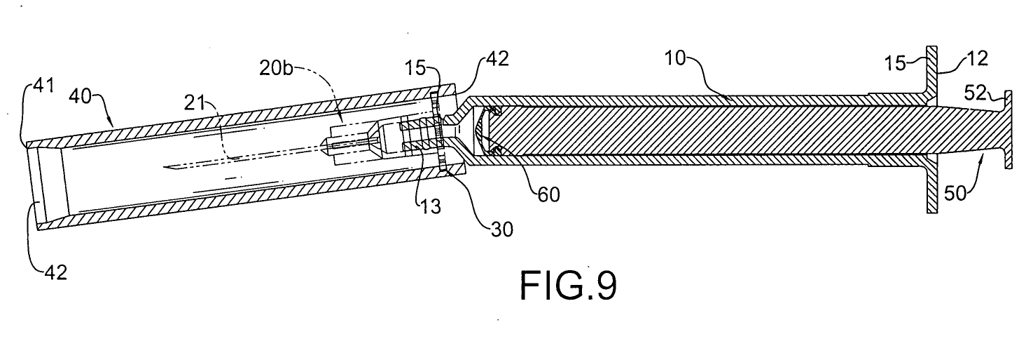

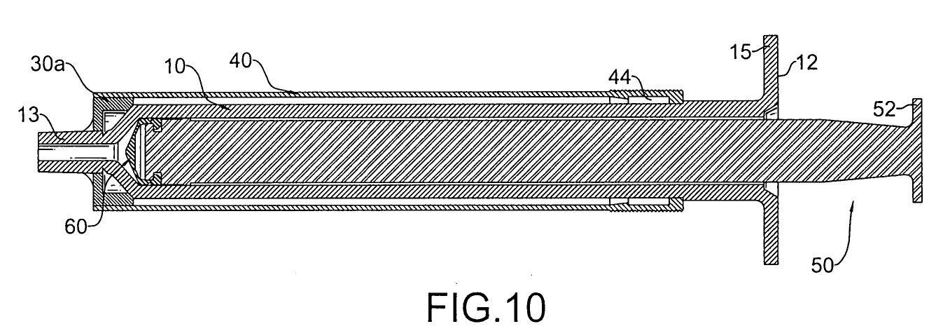

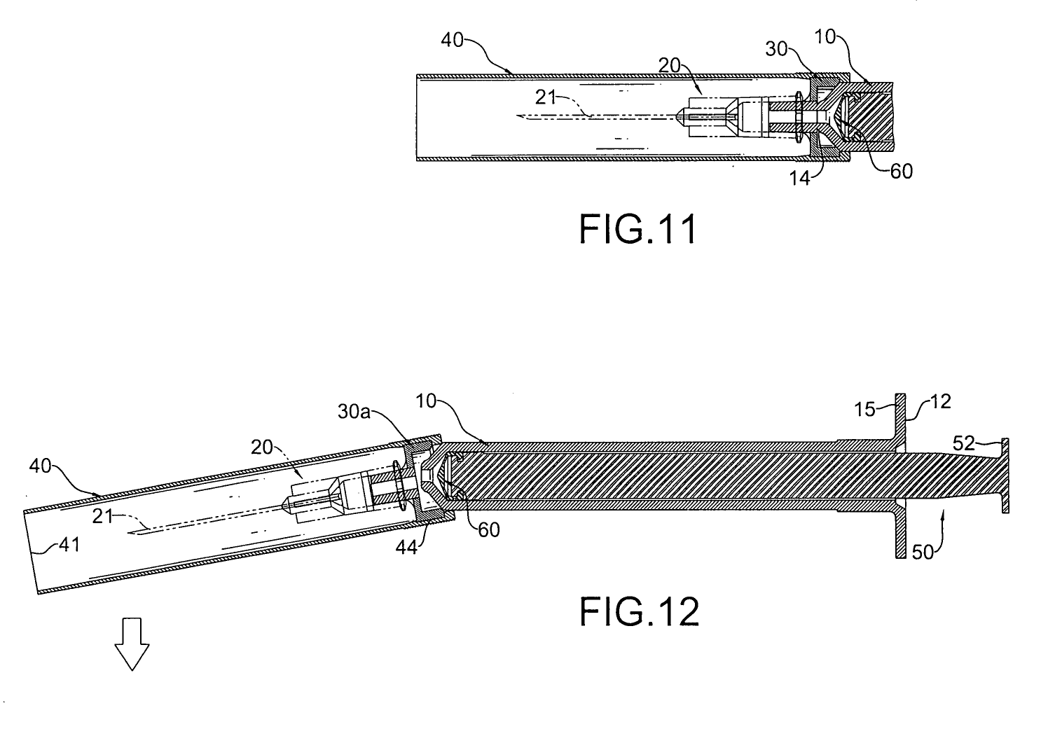

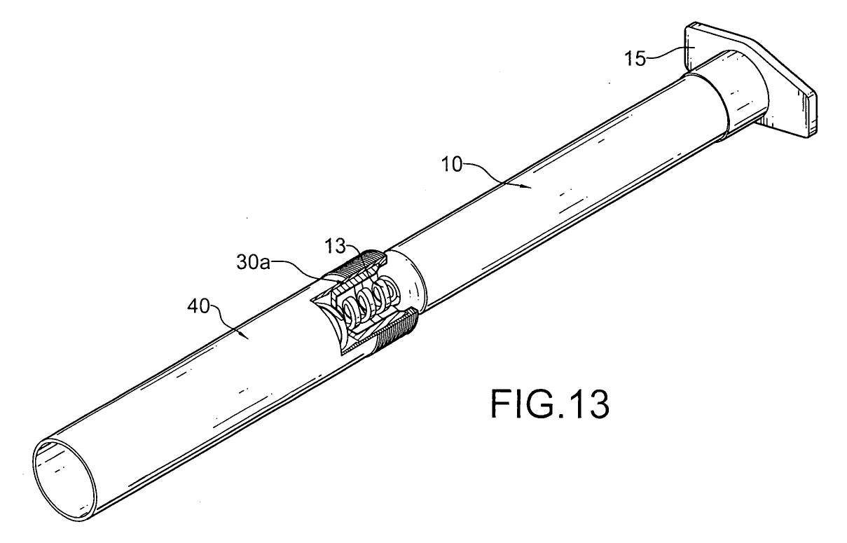

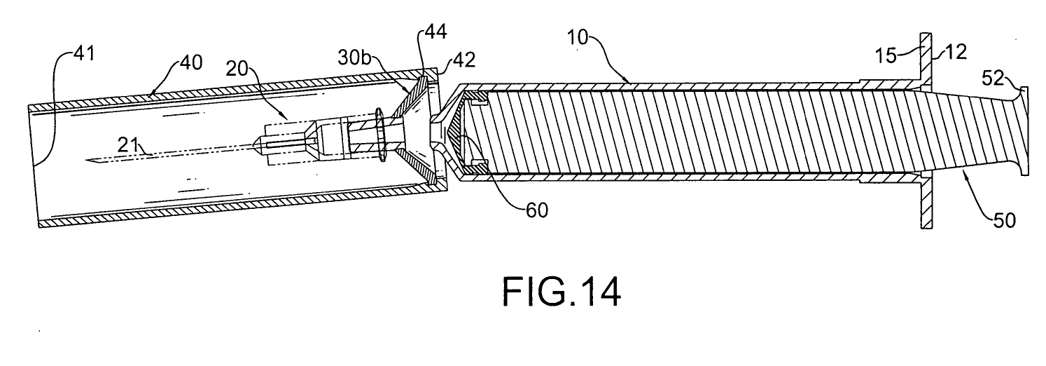

With reference to FIGS. 1, 2, 6 to 14, a safety syringe in accordance with the present invention comprises a syringe body (10), a needle hub (20, 20a, 20b), a casing (40), a plunger (50), an optional washer (30, 30a, 30b) and an optional plug (60).

The syringe body (10) comprises a front end (11), a rear end (12), an outside diameter, a neck (13), a notch (14) and an optional finger flange (15). The neck (13) is formed on and protrudes coaxially from the front end (11) and has a proximal end, a distal end and an outer surface. The notch (14) is formed around the neck (13) in the outer surface near the proximal end and may not be formed completely around the neck (13) so the neck (13) does not detach completely from the syringe body (10) after breaking the notch (14). The finger flange (15) may be oval, allows the syringe body (10) to be held securely during an injection, is formed on and protrudes radially out from the rear end (12).

The needle hub (20, 20a, 20b) is mounted on the distal end of the neck (13), may screw onto, clamp onto, glue to, weld to or stick on the neck (13) and comprises a hub and a needle (21). The needle (21) is hollow and is mounted in and protrudes coaxially from the hub.

The washer (30, 30a, 30b) is mounted around the neck (13), may be annular a tubular or conical and comprises a center, an outer edge, a through hole (31) and multiple optional protrusions (32). Preferably, the washer (30) is alignment. The through hole (31) is formed through the center and is mounted around the neck (13) between the front end (12) of the syringe body (10) and the needle hub (20). The protrusions (32) are formed on and protrude out from the outer edge.

The casing (40) is a hollow tube, is mounted slidably around the syringe body (10) and comprises a front end (41), a rear end (42), an inside diameter, an inner surface, an annular groove (44, 44′) and multiple optional longitudinal. grooves (43). The inside diameter of the casing (40) corresponds to the outside diameter of the syringe body (10). The annular groove (44, 44′) is formed in the inner surface near the rear end (42), holds the washer (30, 30a, 30b) and has a width. The width of the annular groove (44) may be constant. However, the width of the groove (44′) may be narrow at one point and wide opposite to that point so the washer (30, 30a, 30b) inclines when the neck is broken at the notch (14). The longitudinal grooves (43) are formed in the inner surface, communicate with the front end (41), correspond respectively to and slidably hold the protrusions (32) of the washer (30).

The plunger (50) is mounted slidably in the syringe body (10), may be a rod and has a front end, a rear end, a tip (51) and a head (52). The tip (51) is formed on the front end. The head (52) is formed on and protrudes radially out from the rear end. Pushing the head (52) ejects a liquid from the syringe body (10).

The plug (60) is mounted on the tip (51) of the plunger (50) and seals the syringe body (10) so liquid in front of the plug (60) does not leak from the rear end (12) of the syringe body (10).

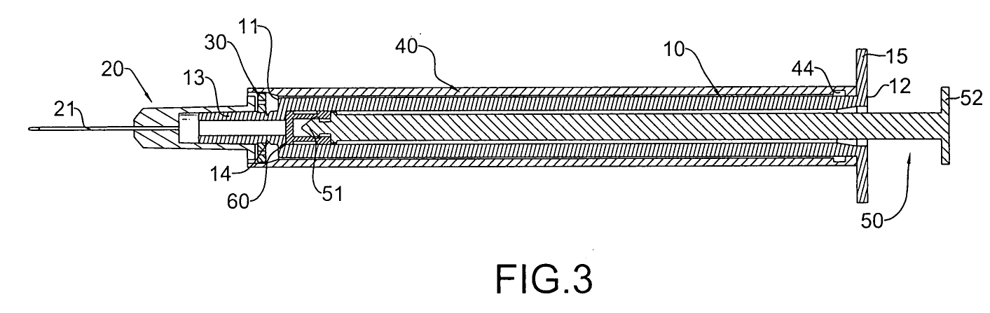

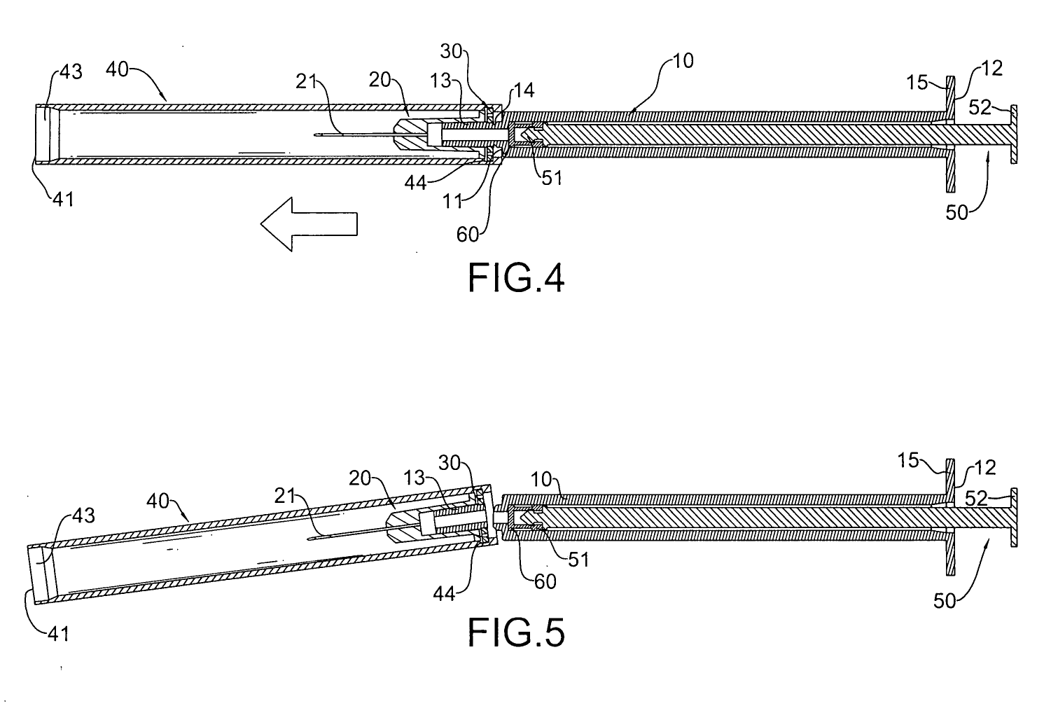

With further reference to FIGS. 3 to 5, 6, 7, 9, 12 and 14, after injecting, a user pulls the body (10) out of the rear end (42) of the casing (40) until the washer (30, 30a, 30b) seats in the annular groove (44, 44′) near the rear end (42) of the casing (40). With the needle hub (20, 20a, 20b) inside the casing (40), the user bends the syringe body (10) relative to the casing (40) until the neck (13) breaks at the notch (14). The washer (30, 30a, 30b) held in the annular groove (44, 44′) holds the needle hub (20, 20a, 20b) with the needle (21) in the casing (40). Therefore, used needle (21) will not prick the user because the used needle (21) is held inside the casing (40) and a cap does not have to be placed over the needle (21).

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A safety syringe comprising:
a syringe body comprising
a front end;
a rear end;
an outside diameter;
a neck formed on and protruding coaxially from the front end and having
a proximal end;
a distal end; and
an outer surface; and
a notch formed around the neck in the outer surface near the proximal end;
a needle hub mounted on the distal end of the neck and comprising
a hub; and
a needle being hollow and mounted in and protruding coaxially from the hub;
a casing being a hollow tube, mounted slidably around the syringe body and comprising
a front end;
a rear end;
an inside diameter corresponding to the outside diameter of the syringe body;
an inner surface; and
an annular groove formed in the inner surface near the rear end and having a width; and
a plunger mounted slidably in the syringe body and comprising
a front end;
a rear end;
a tip formed on the front end; and
a head formed on and protrudes radially out from the rear end.

2. The safety syringe as claimed in claim 1, wherein the safety syringe further comprises a washer mounted around the neck and comprising
a center;
an outer edge; and
a through hole formed through the center and mounted around the neck between the front end of the syringe body and the needle hub.

3. The safety syringe as claimed in claim 2, wherein the washer is annular.

4. The safety syringe as claimed in claim 3, wherein the washer is alignment.

5. The safety syringe as claimed in claim 2, wherein the washer is tubular.

6. The safety syringe as claimed in claim 2, wherein the washer is conical.

7. The safety syringe as claimed in claim 4, wherein
the washer further comprises multiple protrusions formed on and protruding out from the outer edge;
the casing further comprises multiple longitudinal grooves formed in the inner surface, communicating with the front end, corresponding respectively to and slidably holding the protrusions of the washer.

8. The safety syringe as claimed. in claim 1, wherein the notch is formed incompletely around the neck.

9. The safety syringe as claimed in claim 1, wherein the width of the annular groove is constant.

10. The safety syringe as claimed in claim 1, wherein the width of the annular groove is narrow at one point and wide opposite to that point.

11. The safety syringe as claimed in claim 1, wherein the needle hub screws onto the neck of the syringe body.

12. The safety syringe as claimed in claim 1, wherein the needle hub clamps onto the neck of the syringe body.

13. The safety syringe as claimed in claim 1, wherein the needle hub sticks on the neck of the syringe body.

14. The safety syringe as claimed in claim 1, wherein the needle hub glues to the neck of the syringe body.

15. The safety syringe as claimed in claim 1, wherein the needle hub welds to the neck of the syringe body.

16. The safety syringe as claimed in claim 1, wherein the plunger is a rod.

17. The safety syringe as claimed in claim 1, wherein the syringe body further comprises a finger flange formed on and protruding radially out from the rear end.

18. The safety syringe as claimed in claim 17, wherein the finger flange is oval.

19. The safety syringe as claimed in claim 1, wherein the safety syringe further comprises a plug mounted on the tip of the plunger and sealing the syringe body.
